(19) United States
(12) Patent Application Publication
(10) Pub. No.: US 2006/0144292 A1
(43) Pub. Date: Jul. 6, 2006

(54) High-solids content, pourable anti-settling thickening agent

(76) Inventors: Wayne Mattingly, Louisville, KY (US); Alan Steinmetz, Louisville, KY (US)

Correspondence Address: SUD-CHEMIE INC., 1600 WEST HILL STREET, LOUISVILLE, KY, 40210, US

(21) Appl. No.: 11/322,472

(22) Filed: Dec. 30, 2005

Related U.S. Application Data
(63) Continuation-in-part of application No. 10/756,595, filed on Jan. 13, 2004.

(51) Int. Cl. C08L 91/00; C07D 295/18; C08L 91/06

(52) U.S. Cl. 106/268; 106/271; 106/316

(57) ABSTRACT

The present development is an improved rheological additive for coating compositions comprising an organic solvent, a hydrocarbon wax thickener, and a dispersing agent. The dispersing agent is selected from oxidized, hyper-branched polyalphaolefins, polymeric amide-based hyperdispersants, and unsaturated fatty acids, and combinations thereof. The rheological additive can have a solids content of up to about 31 wt %. Further, these high solids content pourable additives can be prepare without adding water to the compositions.

### CROSS-REFERENCE TO RELATED APPLICATIONS

The present application is related to U.S. application Ser. No. 10/756,595 filed on Jan. 13, 2004 and incorporated herein in its entirety by reference.

### BACKGROUND

The present development relates to an improved rheological additive for coating compositions. Specifically, a rheological additive is described that comprises an organic solvent at concentrations of greater than about 68 wt % and a hydrocarbon wax thickener at concentrations of from about 20 wt % to about 31 wt %, and a dispersing agent at concentrations of up to about 5 wt %. The resulting product is a viscous liquid with a relatively high solids content that can be used in coating compositions, and even in applications that require an essentially water-free coating.

It is well known in the art that rheological thickeners can be added to paints and similar coatings to modify the viscosity of the coating and to maintain the suspension of pigments used, for example, to give color to the coating. Historically, these rheological thickeners were prepared by combining organic solvents with about 25 wt % to about 40 wt % oxidized polyethylene wax to form a solid paste. The paste hardness increased with higher wax concentrations. However, the resulting thickeners were difficult to handle and did not disperse well in the coatings.

By adding sulfated castor oil with the oxidized polyethylene wax and processing the material through a scraped-surface heat exchanger, softer high solids (30 wt % to about 40 wt %) wax dispersions can be prepared. The resulting additive does disperse more easily in coatings than the pastes, but the additive has a grease-like consistency and is not pourable.

Pourable additives that are easier to handle and that disperse in the coatings rapidly have been prepared by combining relatively low concentrations of oxidized polyethylene wax with organic solvents and, optionally, sulfated castor oil or similar surfactants. A scraped-surface heat exchanger is used for cooling the dispersion during the manufacturing process. Although easy to use, these pourable additives typically have a total solids content of about 20 wt %, wherein the wax provides only a portion of the total solids, commonly in the range of about 17 wt %, with the balance of the solids resulting from byproducts such as sulfated castor oil. Further, these pourable additives often comprise at least small quantities of water, which may be undesirable in certain applications.

### SUMMARY OF THE INVENTION

The present development relates to an improved rheological additive for coating compositions. The rheological additive comprises an organic solvent, a hydrocarbon wax thickener, and a dispersing agent. An exemplary organic solvent for the additive is a relatively inexpensive, environmentally-friendly, non-aromatic solvent, such as mineral spirits. The dispersing agent is selected from oxidized, hyper-branched polyalphaolefins, polymeric amide-based hyperdispersants, and unsaturated fatty acids. The rheological additive can have a solids content of up to about 31 wt %. Further, these high solids content pourable additives can be prepare without adding water to the compositions.

### DETAILED DESCRIPTION OF THE PREFERRED EMBODIMENT

Rheological thickeners are added to paints and similar coatings to modify the viscosity of the coating and to maintain the suspension of pigments used, for example, to give color to the coating. The most preferable thickeners demonstrate both anti-sag and anti-settling properties while also demonstrating good flow properties, or thickeners that can deliver a fairly high viscosity at a very low shear rate. However, the viscosity needed to maintain the suspension can create problems when the coating is to be applied to a surface. In the latter case, a balance must be achieved so that the product has a high enough viscosity to prevent sag, but the product must also be able to flow adequately to allow the coating to be easily and evenly dispersed.

The rheological additive of the present invention is intended for use in paints and similar coatings. The additive comprises an organic solvent present at a concentration of greater than about 68% by weight and a hydrocarbon wax present at a concentration of about from 20% by weight to about 31% by weight and a dispersing agent present at a concentration of up to about 5% by weight. The organic solvent is preferably a relatively inexpensive, environmentally-friendly, non-aromatic solvent, such as mineral spirits. Exemplary waxes that can be used, without limitation, include oxidized polyethylene wax. The dispersing agent is preferably selected from the group consisting of oxidized, hyper-branched polyalphaolefins, polymeric amide-based hyperdispersants, and unsaturated fatty acids.

It has been surprisingly found that when a dispersing agent selected from the group consisting of oxidized, hyper-branched polyalphaolefins, polymeric amide-based hyperdispersants, and unsaturated fatty acids is added to a mineral spirits solvent further comprising a hydrocarbon wax, a pourable additive is formed even at relatively high concentrations of wax addition. Polyalphaolefins that are molecular weight polymers with acid numbers greater than about 60 have been found to be more effective than polyalphaolefins with acid numbers of about 50 or lower. Thus, the additive of the present invention comprises a solids concentration directly due to the hydrocarbon wax, and not contaminated with surfactant byproducts.

The additive is prepared by quickly adding the wax to the solvent combined with dispersant, and carefully maintaining the reaction temperature for a predetermined period of time. The temperature is then slowly decreased to a temperature of about 75° C. Then the mixture may be fed through a scraped-surface heat exchanger, such as a Votator, to drop the temperature to from about 25° C. to about 30° C., and the additive can then be packaged; or, alternatively, the mixture may remain in the mix tank under agitation where it is allowed to cool to a temperature of from about 25° C. to about 30° C., with or without the use of a cooling jacket, and then the additive can be pumped directly into drums.

In a representative embodiment, a rheological additive comprises from about 68 wt % to about 80 wt % mineral spirits and from about 20 wt % to about 31 wt % oxidized polyethylene wax and up to about 5 wt % oxidized, hyper-branched polyalphaolefin, wherein the oxidized polyethylene wax and the polyalphaolefin together comprise up to about 31 wt % of the additive. The additive is prepared by charging the mineral spirits into a reaction tank that has been cleaned and dried with nitrogen. The mineral spirits is either added by vacuum or it can be pumped in using an air pump. A nitrogen sparge is started and high agitation. The dispersant—the polyalpholefin—is then added and mixed into the mineral spirits. The wax is quickly charged into the mineral spirit/dispersant mixture through a funnel or manway. When the wax is completely charged, the reactor is sealed and vented, and is then heated to a temperature of from about 110° C. to about 115° C. The elevated temperature is maintained until the wax is completely melted and the mixture is homogeneous. A typical reaction time at the elevated temperature is about 30 minutes. When the mixture is homogeneous, the temperature is allowed to slowly decrease to from about 75° C. to about 80° C. using a cooling water heat exchanger. The temperature should decrease from the 110° C. reaction temperature to about 75° C. within about 2 hours. After the mixture reaches about 75° C., the agitation is decreased and the mixture is fed to a Votator, or similar scraped-surface heat exchanger, having an outlet temperature set at about 25° C. The product exits the Votator into drums.

The concentration polyalphaolefin must be controlled to prevent settling due to use of too low a concentration of polyalphaolefin versus gelation, which can be caused by the use of too high a concentration of polyalphaolefin. For example, if a polyalphaolefin with molecular weight polymers of C30+ and an acid number of about 88, such as X-6112 (available from Baker Petrolite) is combined with an oxidized polyethylene wax, such as A-C 629 (available from Honeywell International) in a mineral spirits solvent to yield about 30 wt % solids in the rheological additive, the polyalphaolefin concentration is preferably about 1.4 wt % and the oxidized polyethylene concentration is preferably about 28.6 wt % in order to obtain a stable suspension. In the absence of the polyalphaolefin, e.g. when the oxidized polyethylene concentration is about 30.0 wt %, a soft paste is formed by the oxidized polyethylene in mineral spirits. When the polyalphaolefin concentration is greater than about 1.6 wt % and the oxidized polyethylene concentration is less than about 28.4 wt % in a mineral spirit solvent, the resulting product demonstrates unacceptable gelation. However, the relative concentration of polyalphaolefin and oxidized polyethylene will vary somewhat for each application depending on the polyalphaolefin selected, the oxidized polyethylene selected, and the targeted stability of the suspension.

Similarly, a rheological additive comprising from about 68 wt % to about 80 wt % mineral spirits and from about 20 wt % to about 31 wt % oxidized polyethylene wax and up to about 2 wt % polymeric amide-based hyperdispersants or unsaturated fatty acids may be prepared by the method described above, wherein the polyalphaolefin is replaced by the polymeric amide-based hyperdispersant or unsaturated fatty acid. And more preferably, a rheological additive comprising from about 68 wt % to about 72 wt % mineral spirits and from about 28 wt % to about 31 wt % oxidized polyethylene wax and up to about 2 wt % polymeric amide-based hyperdispersants or unsaturated fatty acids may be prepared by the method described above, wherein the polyalphaolefin is replaced by the polymeric amide-based hyperdispersant or unsaturated fatty acid. A representative example, without limitation, of a hyperdispersant that may be used in the additive of the present development is Solsperse 13940 (available from Lubrizol). A representative example, without limitation, of a unsaturated fatty acid that may be used in the additive of the present development is oleic acid.

The rheological additive of the present invention can be used in paints and other coating compositions. The additive differs from the prior art by being formulated and prepared such that the additive is essentially free of water, and is pourable, and has a sufficiently high wax content that the additive can provide viscosity to the coating compositions at very low shear rates, thereby providing anti-settling characteristics to the coating compositions. Further, the additive of the present invention as prepared with the dispersing agents demonstrates efficiency in a coating formulation at least as effective as coating formulations using more expensive oxidized copolymer hydrocarbon wax additives. This can result in improved anti-settling and anti-sagging properties in the coating product.

It is understood that the composition of the rheological additive and the specific processing conditions may be varied within limits without exceeding the scope of this development.

What is claimed is:

1. A rheological additive comprising an organic solvent and a hydrocarbon wax thickener and a dispersing agent selected from the group consisting of oxidized hyper-branched polyalphaolefins, polymeric amide-based hyperdispersants, unsaturated fatty acids, and combinations thereof.

2. The additive of claim 1 wherein said organic solvent is mineral spirits.

3. The additive of claim 1 wherein said hydrocarbon wax is an oxidized polyethylene wax.

4. The additive of claim 1 wherein said organic solvent is present at a concentration of greater than about 68% by weight and said hydrocarbon wax is present at a concentration of about from 20% by weight to about 31% by weight and said dispersing agent is present at a concentration of up to about 5% by weight.

5. The additive of claim 4 wherein said organic solvent is present at a concentration of from 68 wt % to 72 wt % and said hydrocarbon wax is present at a concentration of from 28% by weight to about 31% by weight and said dispersing agent is present at a concentration of up to about 2% by weight.

6. A rheological additive comprising an organic solvent and a hydrocarbon wax thickener and an oxidized hyper-branched polyalphaolefins.

7. The additive of claim 6 wherein said organic solvent is mineral spirits.

8. The additive of claim 6 wherein said hydrocarbon wax is an oxidized polyethylene wax.

9. The additive of claim 6 wherein said organic solvent is present at a concentration of is present at a concentration of from 68 wt % to 80 wt % and said hydrocarbon wax is present at a concentration of from 20% by weight to about 30% by weight and said oxidized hyper-branched polyalphaolefins is present at a concentration such that the total weight percent of the hydrocarbon wax plus the oxidized hyper-branched polyalphaolefins equals about 31 wt %.

10. The additive of claim 6 wherein said organic solvent is present at a concentration of is present at a concentration of from 68 wt % to 72 wt % and said hydrocarbon wax is present at a concentration of from 28% by weight to about 29% by weight and said oxidized hyper-branched polyalphaolefins is present at a concentration such that the total weight percent of the hydrocarbon wax plus the oxidized hyper-branched polyalphaolefins equals about 30 wt %.

11. The additive of claim 6 wherein said additive is prepared by combining said organic solvent with said oxidized hyper-branched polyalphaolefins in a mix tank, and then charging said wax into said mix tank with agitation to form an additive mixture, and maintaining a predetermined reaction temperature for a predetermined period of time, and then decreasing the temperature of said additive mixture to a temperature of from about 25° C. to about 30° C.

12. The additive of claim 11 wherein the temperature of said additive mixture is decreased to from about 25° C. to about 30° C. by feeding said additive mixture through a scraped-surface heat exchanger.

13. The additive of claim 11 wherein the temperature of said additive mixture is decreased to from about 25° C. to about 30° C. by allowing said mixture to cool in said mix tank under agitation.

14. A rheological additive comprising an organic solvent and a hydrocarbon wax thickener and an adjunct selected from polymeric amide-based hyperdispersants and unsaturated fatty acids.

15. The additive of claim 14 wherein said organic solvent is mineral spirits.

16. The additive of claim 14 wherein said hydrocarbon wax is an oxidized polyethylene wax.

17. The additive of claim 14 wherein said organic solvent is present at a concentration of is present at a concentration of from 68 wt % to 80 wt % and said hydrocarbon wax is present at a concentration of from 20% by weight to about 30% by weight and said adjunct is present at a concentration such that the total weight percent of said hydrocarbon wax plus said adjunct equals about 31 wt %.

18. The additive of claim 14 wherein said organic solvent is present at a concentration of is present at a concentration of from 69.5 wt % to 70.5 wt % and said hydrocarbon wax is present at a concentration of from 28% by weight to about 29% by weight and said adjunct is present at a concentration such that the total weight percent of said hydrocarbon wax plus said adjunct equals about 30 wt %.

19. The additive of claim 14 wherein said adjunct is oleic acid.

20. The additive of claim 14 wherein said additive is prepared by combining said organic solvent with said oxidized hyper-branched polyalphaolefins in a mix tank, and then charging said wax into said mix tank with agitation to form an additive mixture, and maintaining a predetermined reaction temperature for a predetermined period of time, and then decreasing the temperature of said additive mixture to a temperature of from about 25° C. to about 30° C.
